(19) United States
(12) Patent Application Publication
(10) Pub. No.: US 2011/0165202 A1
(43) Pub. Date: Jul. 7, 2011

(54) SOLID STATE FORMS OF FOSAMPRENAVIR CALCIUM SALT AND PROCESSES FOR PREPARATION THEREOF

(75) Inventors: Edislav LEKSIC, Zagreb (HR); Dragan SEPAC, Zagreb (HR); Dubravka PAVLICIC, Zagreb (HR)

(73) Assignee: PLIVA HRVATSKA D.O.O., Zagreb (HR)

(21) Appl. No.: 12/986,125

(22) Filed: Jan. 6, 2011

Related U.S. Application Data
(60) Provisional application No. 61/292,959, filed on Jan. 7, 2010.
(60) Provisional application No. 61/298,622, filed on Jan. 27, 2010.
(60) Provisional application No. 61/318,742, filed on Mar. 29, 2010.
(60) Provisional application No. 61/331,002, filed on May 4, 2010.
(60) Provisional application No. 61/417,686, filed on Nov. 29, 2010.

(51) Int. Cl. A61K 9/00; C07F 9/655; A61K 31/665; A61P 31/18

(52) U.S. Cl. 424/400; 549/222; 514/99

(57) ABSTRACT

The present invention relates to solid state forms of fosamprenavir calcium salt, process for preparing said solid state forms, and pharmaceutical compositions thereof.

### CROSS-REFERENCE TO RELATED APPLICATIONS

This application claims the benefit of U.S. Provisional Patent Application Ser. Nos. 61/292,959, filed Jan. 7, 2010; 61/298,622, filed Jan. 27, 2010; 61/318,742, filed Mar. 29, 2010; 61/331,002, filed May 4, 2010; and 61/417,686, filed Nov. 29, 2010, which are incorporated herein by reference.

### FIELD OF THE INVENTION

The present invention relates to solid state forms of fosamprenavir calcium salt, process for preparing said solid state forms, and pharmaceutical compositions thereof.

### BACKGROUND OF THE INVENTION

Fosamprenavir calcium salt, calcium (2R, 3S)-1-(4-amino-N-isobutyl-phenylsulfonamido)-4-phenyl-3-(((S)-tetrahydrofuran-3-yloxy)carbonylamino)butan-2-yl phosphate is a phosphate ester prodrug of the protease inhibitor and antiretroviral drug amprenavir. The human body metabolizes fosamprenavir to form amprenavir, which is the active agent. Fosamprenavir calcium salt of the following formula:

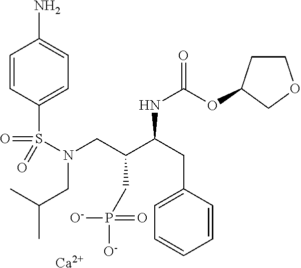

is marketed by GlaxoSmithKline under the trade name Lexiva and Telzir. It is used for HIV treatment.

U.S. Pat. No. 6,436,989 B1 discloses preparation of Fosamprenavir sodium salt. U.S. Pat. No. 6,514,953 B1 discloses polymorphic form I of Fosamprenavir calcium salt. The present invention comprises solid state forms of Fosamprenavir calcium salt.

Polymorphism, the occurrence of different crystal forms, is a property of some molecules and molecular complexes. A single molecule, like Fosamprenavir calcium salt, may give rise to a variety of polymorphs having distinct crystal structures and physical properties like melting point, thermal behaviors (e.g. measured by thermogravimetric analysis—“TGA”, or differential scanning calorimetry—“DSC”), x-ray diffraction pattern, infrared absorption fingerprint, and solid state NMR spectrum. One or more of these techniques may be used to distinguish different polymorphic forms of a compound.

Discovering polymorphic forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need for additional polymorphs of Fosamprenavir calcium salt.

### SUMMARY OF THE INVENTION

In one embodiment, the present invention comprises an amorphous form of Fosamprenavir calcium salt.

In one embodiment, the present invention comprises a rod like amorphous form of Fosamprenavir calcium salt characterized by data selected from: a Scanning Electron Microscope (SEM) image as depicted in FIG. 10, a SEM image as depicted in FIG. 11; a SEM image as depicted in FIG. 12, a SEM image as depicted in FIG. 13, and combinations thereof.

In one embodiment, the present invention comprises an amorphous form of Fosamprenavir calcium salt obtainable by a process comprising heating Fosamprenavir calcium salt, form I, having XRPD peaks at 5.735, 9.945, 11.500, 13.780, 14.930, 15.225, 17.980, 19.745, 21.575, 22.170, 24.505 and 27.020° 2θ±0.2° 2θ.

In one embodiment, the present invention comprises the use of the above described amorphous types of Fosamprenavir calcium salt for the preparation of a formulation.

In another embodiment, the present invention comprises a pharmaceutical composition comprising amorphous types of Fosamprenavir calcium salt described above and at least one pharmaceutically acceptable excipient.

### BRIEF DESCRIPTION OF THE FIGURES

FIG. 1 shows a powder X-ray diffraction (XRD) pattern of amorphous Fosamprenavir calcium obtained according to the procedure described in example 1

FIG. 2 shows a DSC thermogram of amorphous Fosamprenavir calcium obtained according to the procedure described in example 1

FIG. 3 shows a DSC thermogram of amorphous Fosamprenavir calcium obtained according to the procedure described in example 2

FIG. 4 shows a powder XRD pattern of Fosamprenavir calcium form II (peak at 28.49° 2-theta corresponds to Silica powder)

FIG. 5 shows a powder XRD pattern of Fosamprenavir calcium form III (peak at 28.49° 2-theta corresponds to Silica powder)

FIG. 6 shows a powder XRD pattern of amorphous Fosamprenavir calcium obtained according to the procedure described in example 15

FIG. 7 shows a powder XRD pattern of crystalline Form IV of acetone solvate of Fosamprenavir Calcium

FIG. 8 shows a powder XRD pattern of crystalline Form P of Fosamprenavir Calcium

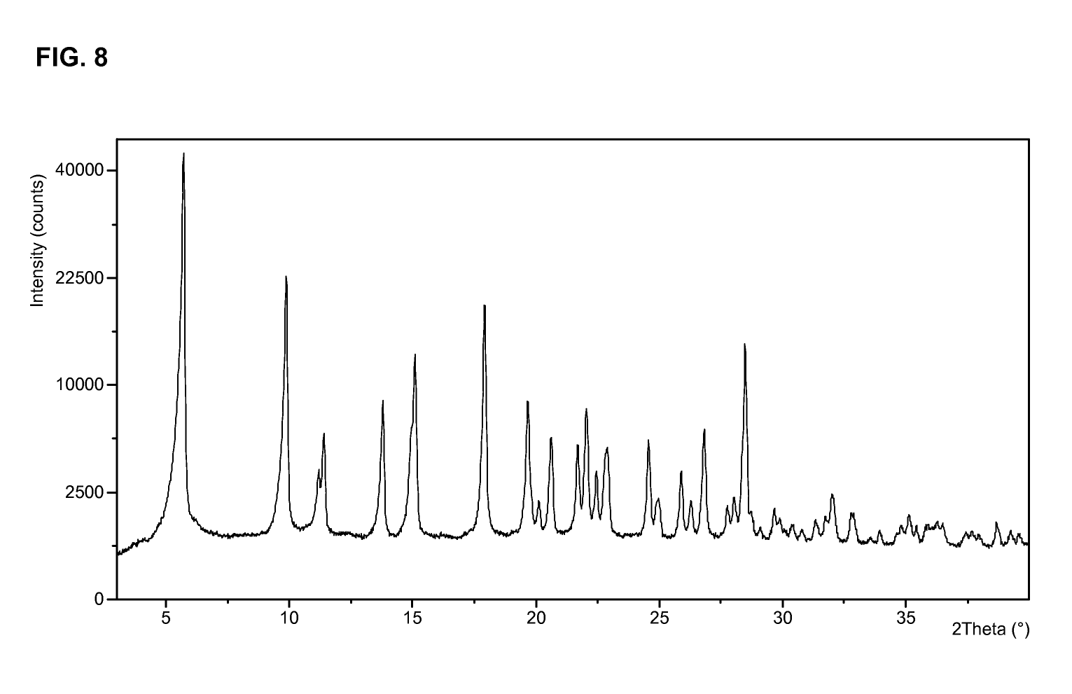

FIG. 9 shows a powder XRD pattern of amorphous Fosamprenavir Calcium obtained according to the procedure described in example 14

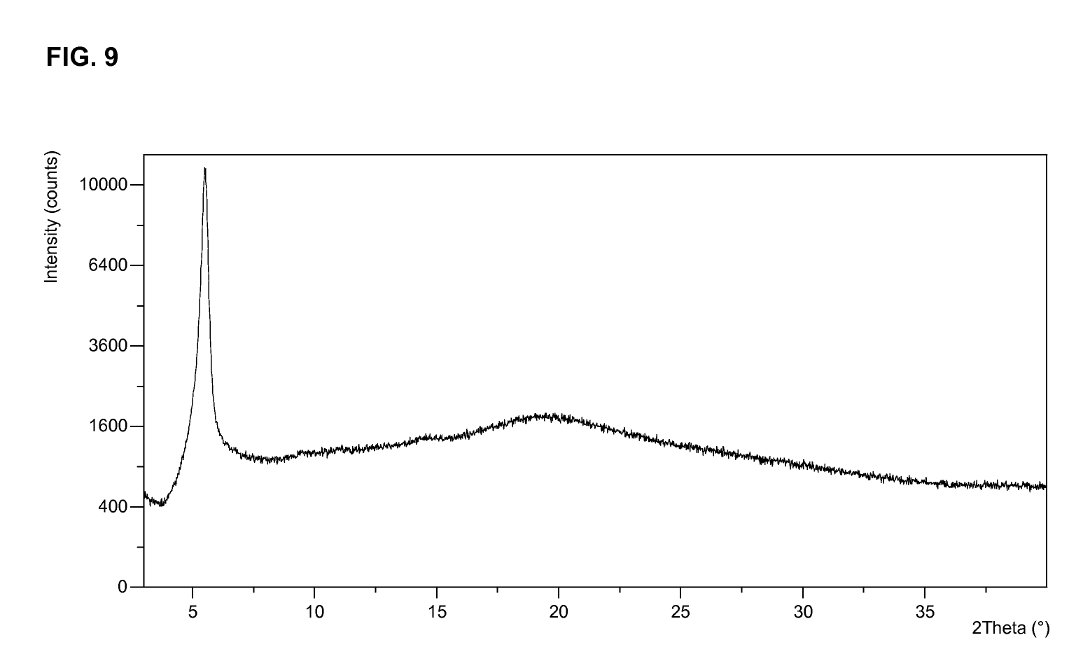

FIG. 10 shows a SEM image of rod like amorphous Fosamprenavir Calcium in magnification 1000× obtained according to the procedure described in example 20

FIG. 11 shows a SEM image of rod like amorphous Fosamprenavir Calcium in magnification 2000× obtained according to the procedure described in example 21

FIG. 12 shows a SEM image of rod like amorphous Fosamprenavir Calcium in magnification 2000× obtained according to the procedure described in example 14

FIG. 13 shows a SEM image of rod like amorphous Fosamprenavir Calcium in magnification 5000× obtained according to the procedure described in example 14

FIG. 14 shows a SEM image of amorphous Fosamprenavir Calcium in magnification 1000× obtained according to the procedure described in example 22

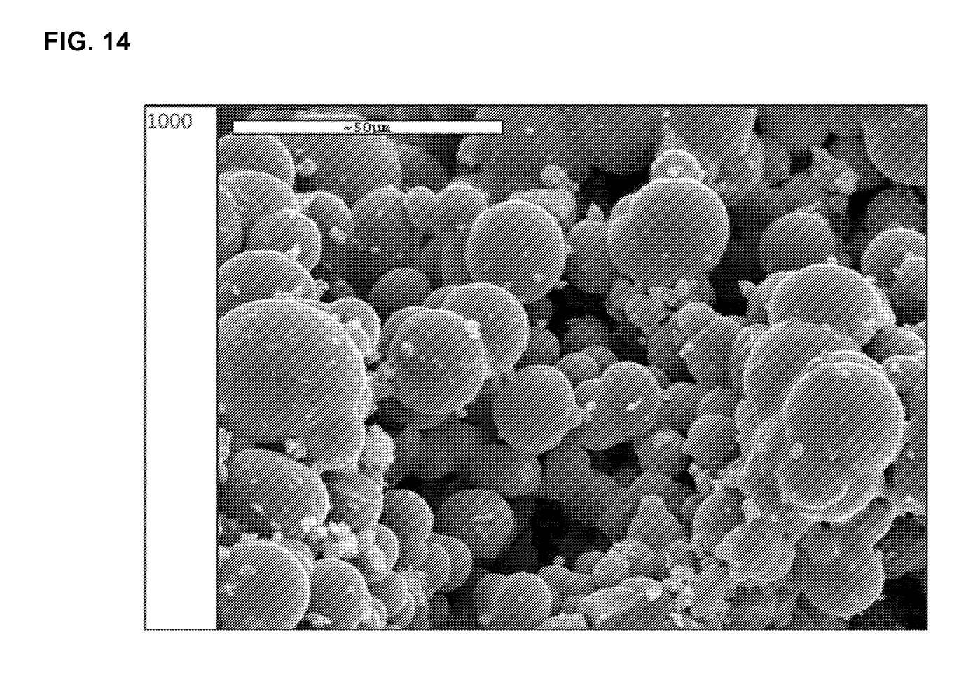

### DETAILED DESCRIPTION OF THE INVENTION

The present invention relates to solid state forms of fosamprenavir calcium salt, processes for preparing said solid state forms, and pharmaceutical compositions comprising one or more of said solid state forms.

A crystal form may be referred to herein as being characterized by graphical data “as shown in,” or “as depicted in” a Figure. Such data include, for example, powder X-ray diffractograms, solid state NMR spectra and DSC thermograms. The skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form, and confirm whether the two sets of data are characterizing the same crystal form or two different crystal forms.

A polymorphic form according to the invention may be referred to herein as “pure” or “polymorphically pure.” This terminology refers to the subject polymorph containing less than about 20% (w/w) of other polymorphic forms. Preferably, when a crystal form according to the invention is referred to as pure or polymorphically pure, it will contain less than 10%, less than 5%, less than 2%, less than 1% or even less than 0.5% of other forms of the compound. In other embodiments, the polymorphs of fosamprenavir calcium according to the invention may contain from 1% to 20% (w/w), from 5% to 20% (w/w), or from 5% to 10% (w/w) of one or more other polymorphic forms of fosamprenavir calcium.

As used herein, the expression “Room temperature” refers to a temperature between about 20° C. and about 30° C. Usually, room temperature ranges from about 20° C. to about 25° C.

As used herein, the term “Overnight” refers to a period of between about 15 and about 20 hours, typically between about 16 to about 20 hours.

As used herein, “Form I” refers to crystalline Fosamprenavir calcium salt having XRPD peaks at 5.735, 9.945, 11.500, 13.780, 14.930, 15.225, 17.980, 19.745, 21.575, 22.170, 24.505 and 27.020° 2θ±0.2° 2θ, as defined in U.S. Pat. No. 6,514,953.

As used herein, the term “Rod like” refers to partices elongated square prisms in a form of a stick.

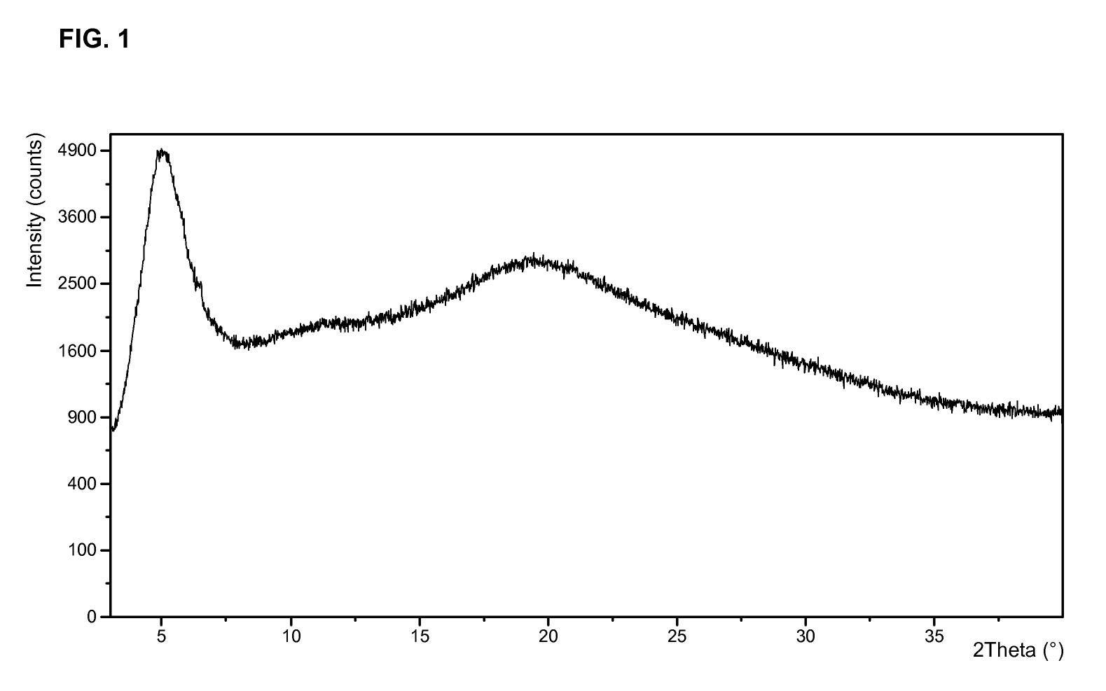

The present invention comprises an amorphous form of Fosamprenavir calcium salt. The amorphous Fosamprenavir calcium salt can be characterized by a powder XRD pattern as depicted in FIG. 1.

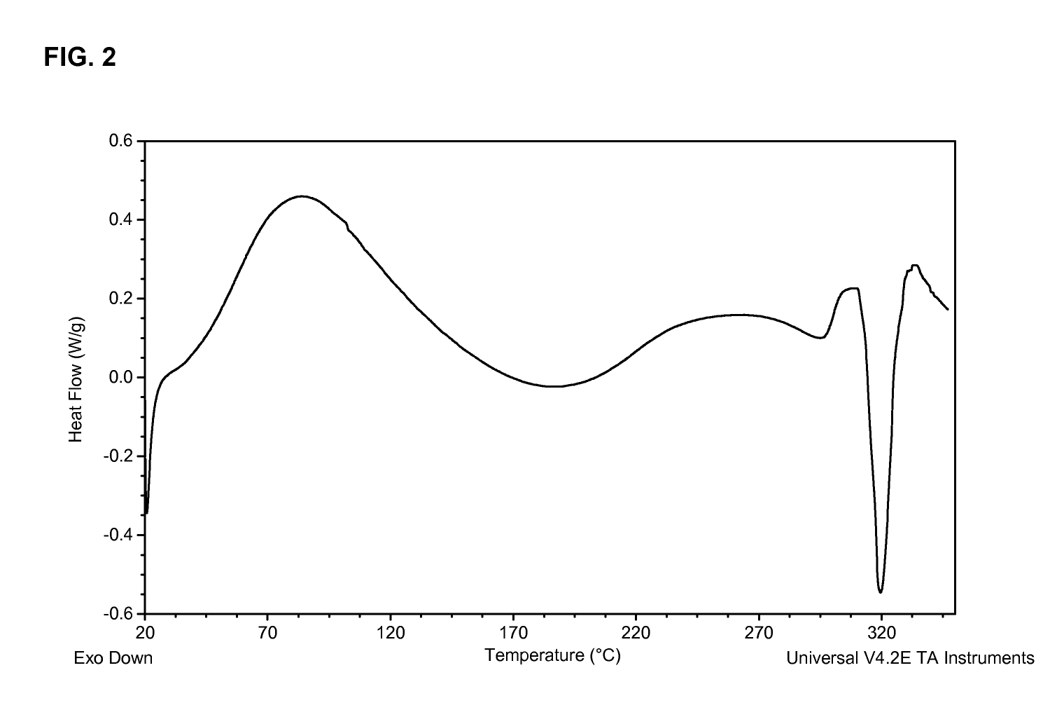

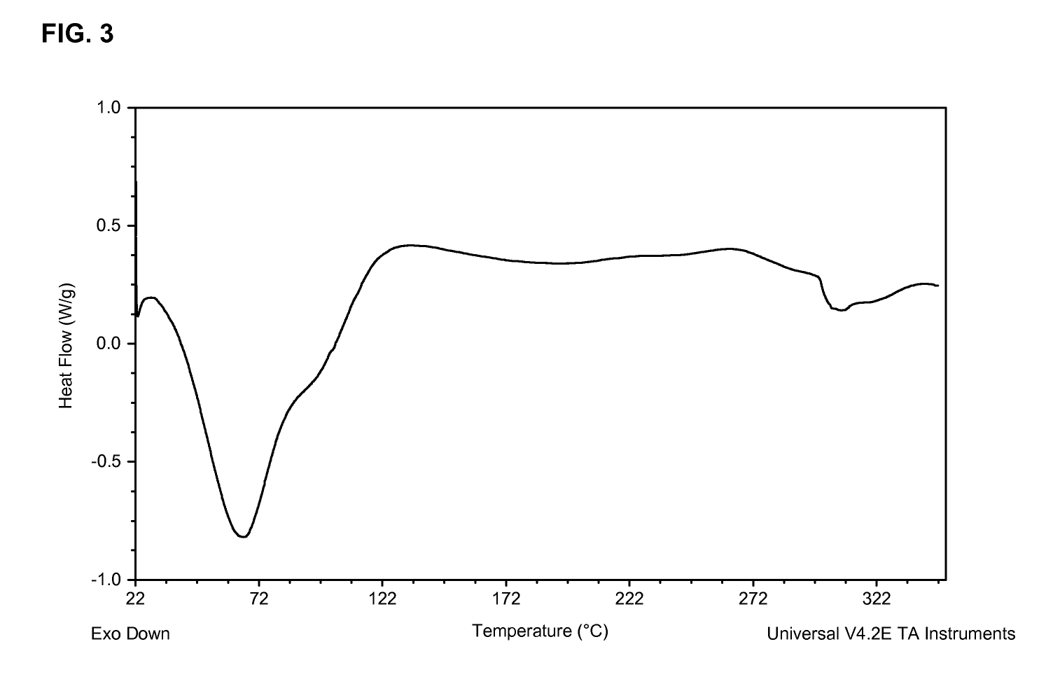

The above amorphous Fosamprenavir calcium salt can be further characterized by data selected from: a DSC thermogram as depicted in FIG. 2; a DSC thermogram as depicted in FIG. 3; and combinations thereof.

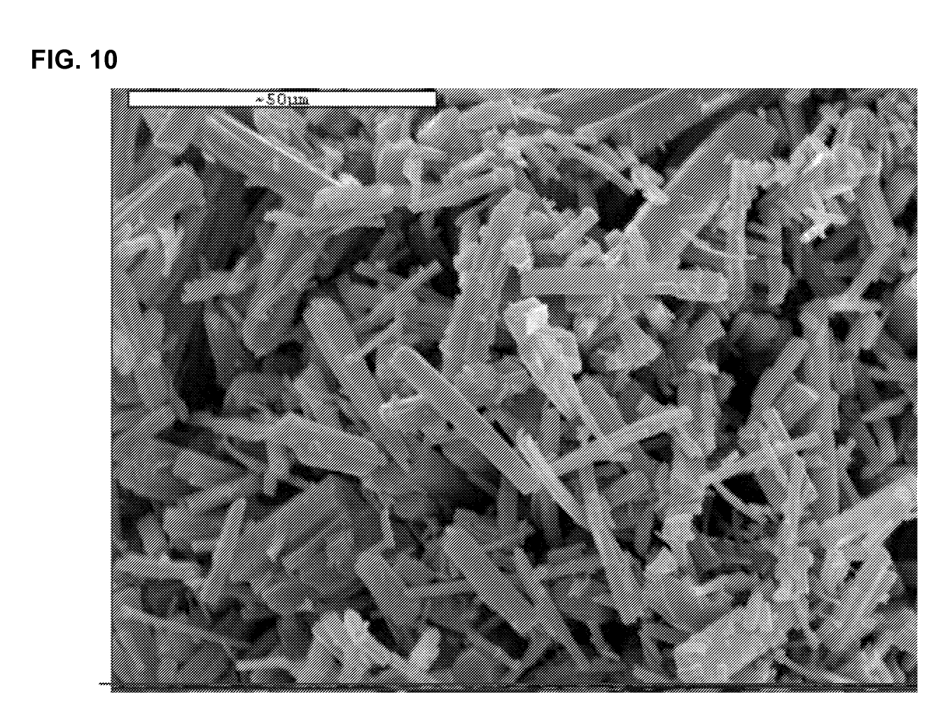

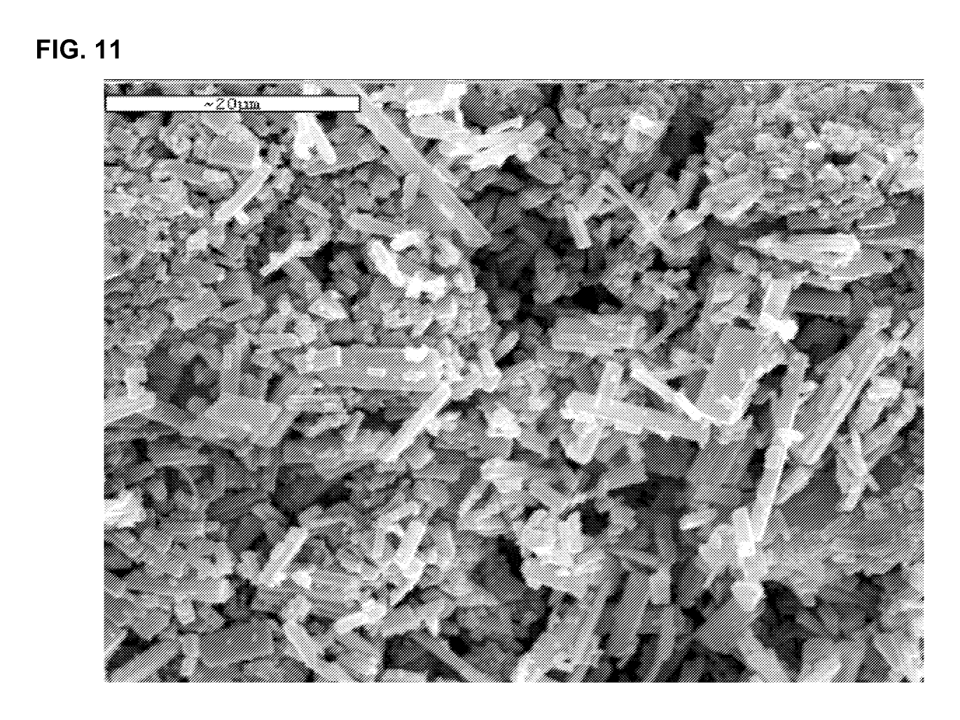

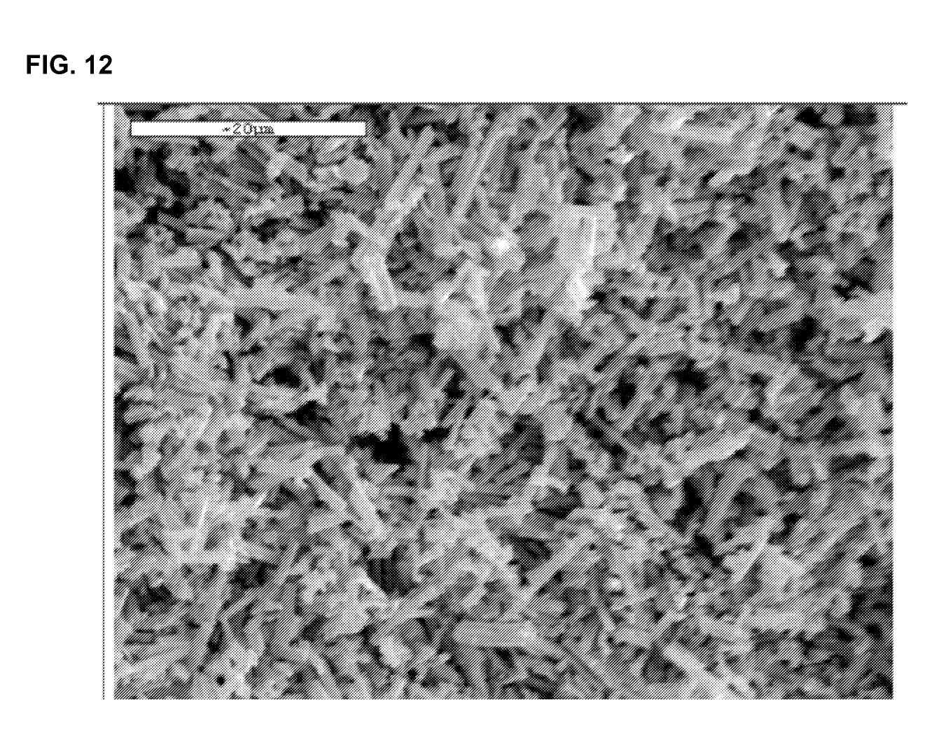

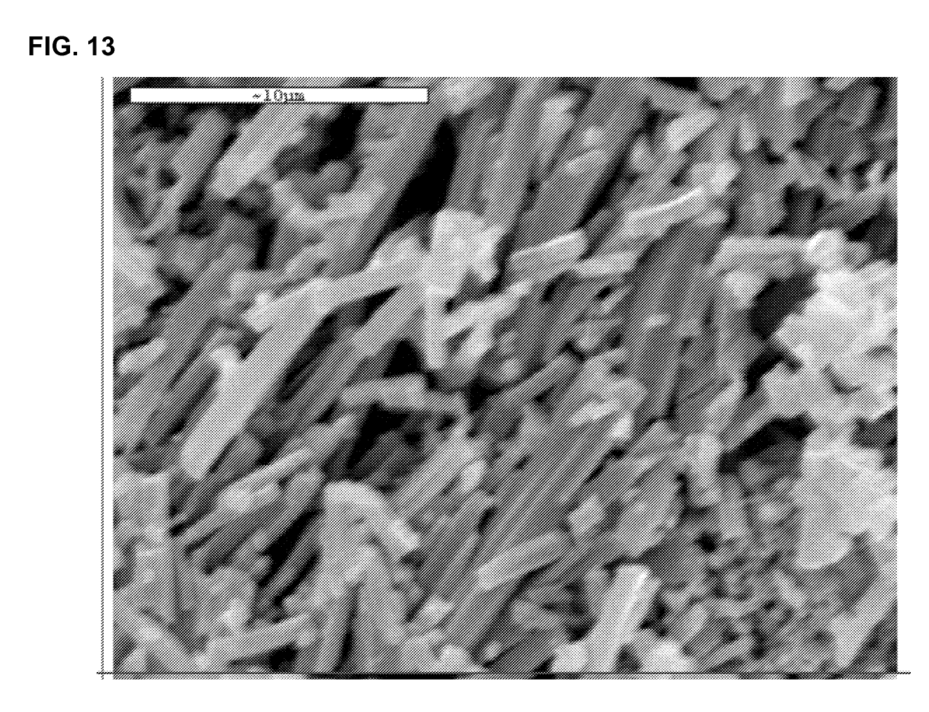

The present invention comprises a rod like amorphous form of Fosamprenavir calcium salt characterized by data selected from: a SEM image as depicted in FIG. 10, a SEM image as depicted in FIG. 11; a SEM image as depicted in FIG. 12, a SEM image as depicted in FIG. 13, and combinations thereof.

A rod like shape is readily distinguished from the typical spherical or irregular shape obtained in a conventional amorphous material by the regular shape of sticks with different lengths.

In one embodiment of the present invention, the particles are obtained as sticks, or rods as observed by SEM imaging. The handling of amorphous powders is known in the art to often be difficult. But, with a particle morphology that is similar to a crystalline form, as described in the present invention, applicants have discovered that it has been possible to avoid some potential technology problems that appear during formulation.

The rod like amorphous particles show optimum bulk powder properties as well as enhanced solubility compared to Form I. Amorphous fosamprenavir calcium comprising rod-like particles prevents material segregation, thus ensuring homogeneity of the material. The larger surface area of this material as compared to amorphous material comprising spherical particles contributes to enhancement of tablet properties such as tablet strength.

The present invention further comprises an amorphous form of Fosamprenavir calcium salt obtainable by a process comprising heating Fosamprenavir calcium salt, form I.

Preferably, the heating is performed, under vacuum, at a temperature of about 45° C., preferably, for a period of about 3 hours.

The process comprises further heating at a temperature of about 70° C. to about 120°, preferably, at a temperature of about 100°, for a period of about 30 minutes to about 5 hours, preferably, 3 hours to about 5 hours, and at a temperature of about 120° to about 130° C., for a period of about 30 minutes to about 2 hours, preferably, for about 2 hours.

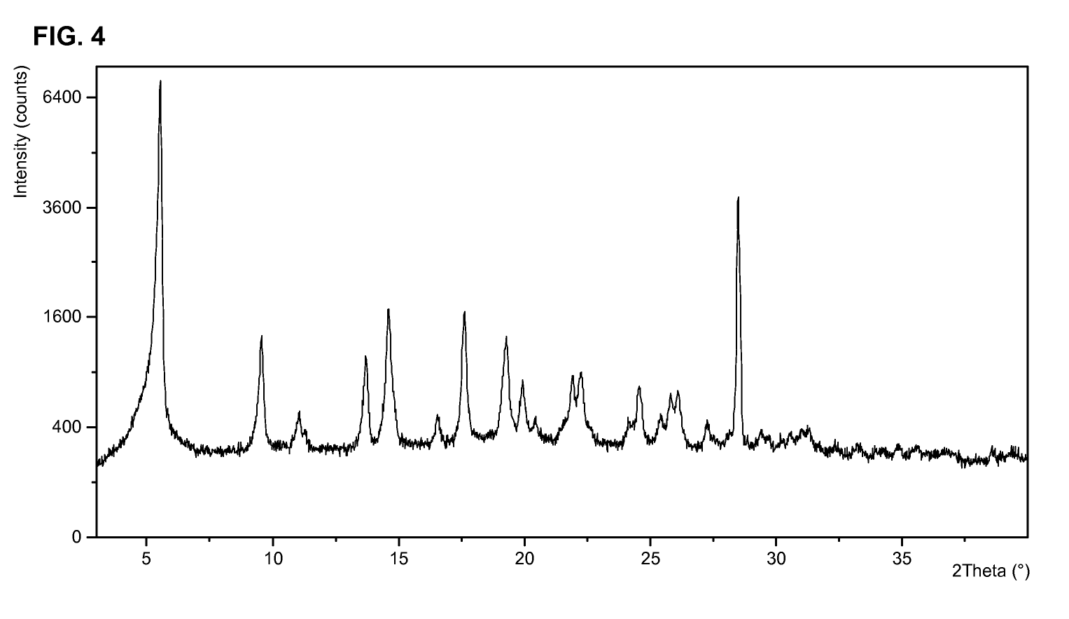

In one embodiment, the present invention comprises a crystalline form of Fosamprenavir calcium salt characterized by data selected from: a powder XRD pattern with peaks at about 14.6, 16.5, 17.6, 19.2 and 25.4° 2θ±0.2° 2θ; a powder XRD pattern as depicted in FIG. 4, and combinations thereof. This form is designated herein as Form II. Fosamprenavir calcium crystalline Form II can be further characterized by additional powder XRD peaks at about 5.5, 9.5, 13.7 19.9 and 26.1° 2θ±0.2° 2θ.

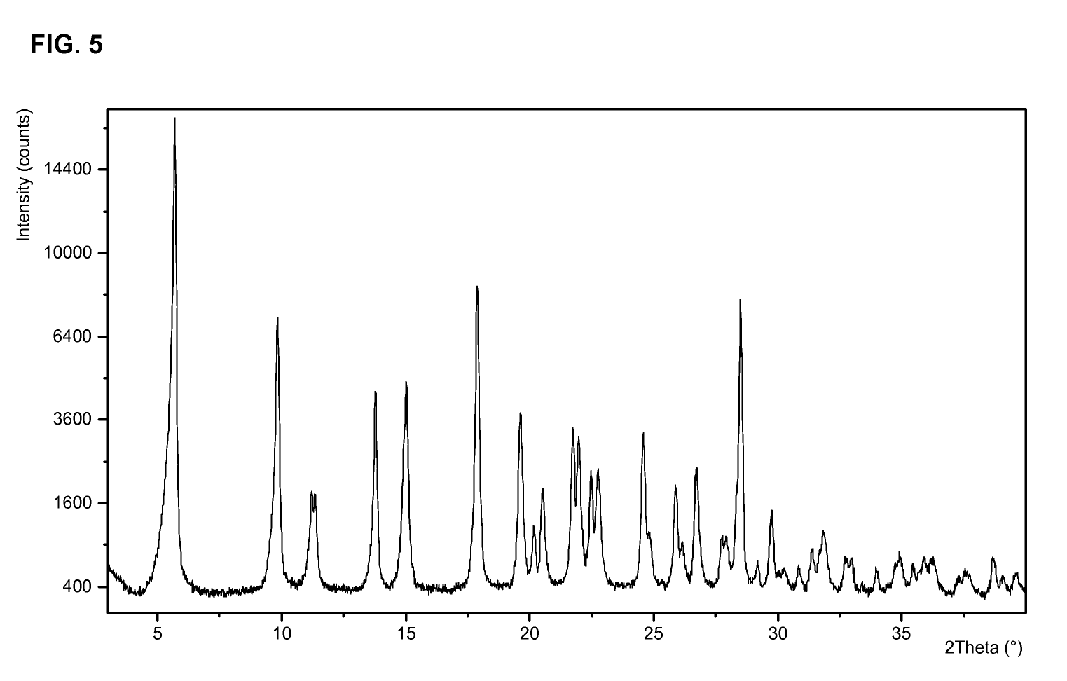

In one embodiment the present invention comprises a crystalline form of Fosamprenavir calcium salt characterized by data selected from: a powder XRD pattern with peaks at about 20.5, 22.0, 22.7 and 29.7° 2θ±0.2° 2θ; a powder XRD pattern as depicted in FIG. 5, and combinations thereof. This form can be designated as form III. The Fosamprenavir calcium crystalline Form III can be further characterized by additional powder XRD peaks at about 5.7, 9.8, 15.0 and 21.7° 2θ±0.2° 2θ.

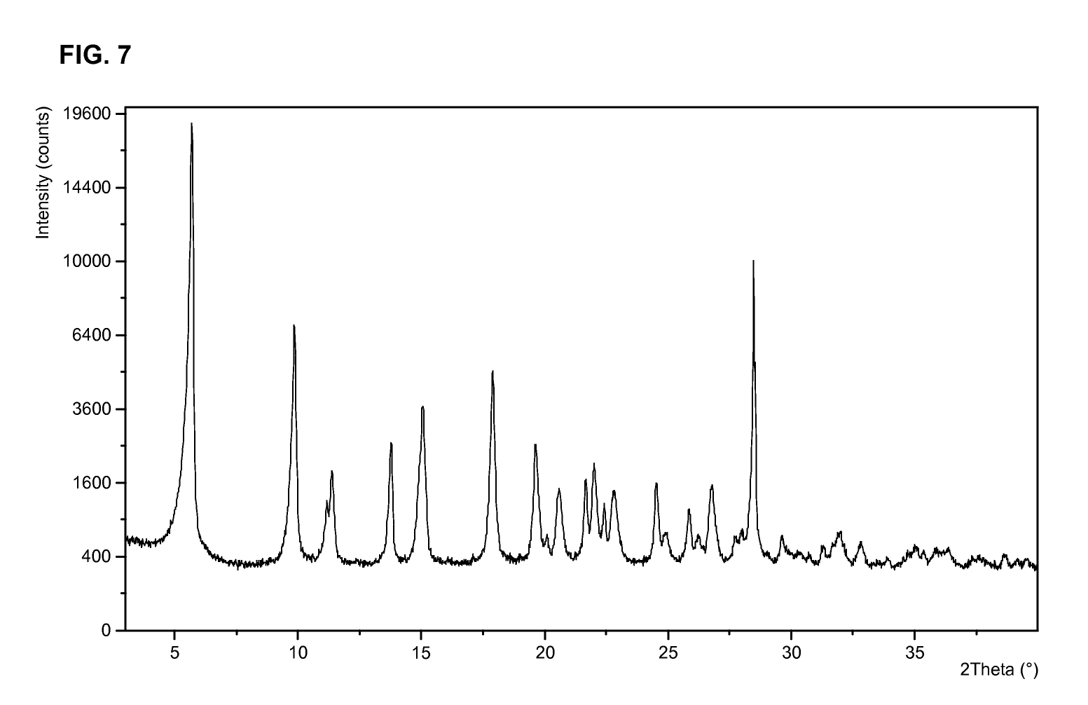

In one embodiment, the present invention comprises a crystalline form of Fosamprenavir calcium salt characterized by data selected from: a powder XRD pattern with peaks at about 20.6, 22.0, 22.8 and 26.2° 2θ±0.2° 2θ; a powder XRD pattern as depicted in FIG. 7, and combinations thereof. This form is designated herein as form IV. Fosamprenavir calcium crystalline form IV of can be further characterized by additional powder XRD peaks at about 5.7, 9.8, 17.9 and 26.8° 2θ±0.2° 2θ. Fosamprenavir calcium crystalline form IV can be an acetone solvate.

In one embodiment, the present invention comprises a crystalline form of Fosamprenavir calcium salt characterized by data selected from: a powder XRD pattern with peaks at about 20.6, 22.0, 22.9, 24.9 and 26.3° 2θ±0.2° 2θ; a powder XRD pattern as depicted in FIG. 4, and combinations thereof. This form can be designated as form P. The Fosamprenavir calcium crystalline Form P can be further characterized by additional powder XRD peaks at about 5.7, 9.9, 28.0 and 29.6° 2θ±0.2° 2θ.

The above described solid state forms of Fosamprenavir calcium salt can be used to prepare formulations by any method known in the art.

In yet another embodiment, the present invention encompasses a pharmaceutical composition comprising the above described solid state forms of Fosamprenavir calcium salt and at least one pharmaceutically acceptable excipient.

The present invention further encompasses 1) a pharmaceutical composition comprising any one or combination of solid state Forms, as described above, and at least one pharmaceutically acceptable excipient; 2) the use of any one or combination of the above-described solid state Forms, in the manufacture of a pharmaceutical composition, and 3) a method of treating HIV. The pharmaceutical composition can be useful for preparing a medicament. The present invention also provides solid state forms as described above for use as a medicament.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### PXRD Method

Samples, after being powdered in a mortar and pestle, were applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngstrom), X'Celerator (2.022° 2Θ) detector. Scanning parameters: angle range: 3-40 deg., step size 0.0167, time per step 37-100 s, continuous scan. The described peak positions were determined by using a silicon powder as an internal standard in an admixture with the sample measured. The position of the silicon (Si) peak was corrected to silicone theoretical peak: 28.45 degrees two theta, and the positions of the measured peaks were corrected respectively. No correction was performed on the presented diffractogram in the FIGS. 2-5.

### DSC Method

DSC analysis was performed on Q 1000 MDSC TA instruments with a heating rate of 10° C./min, under nitrogen flow of 50 ml/min. A standard aluminum, closed pan (with hole) was used, and the sample mass was about 1-5 mg.

### GC Method

Chromatographic parameters

| Capillary column | Phenomenex, ZB-624, 30 m × 0.53 mm, 3.0 μm | | |
|---|---|---|---|
| | or equivalent | | |
| Column temperature | 40° C. isothermal for 7 minutes | | |
| | 40° C. → 220° C. at 15° C./min | | |
| | 220° C. isothermal for 2 minutes | | |
| Injector temperature | 220° C. | | |
| Detector temperature | 250° C. | | |
| Detector | FID | | |
| Carrier | helium (or nitrogen) at 3 mL/min | | |
| Split ratio | 2:1 | | |
| Injection conditions | Vial temperature | 80° | C. |
| for pneumatic | Loop temperature | 90° | C. |
| sampling HS | Transfer line | 100° | C. |
| autosampler | temperature | | |
| | Vial equilibration | 30 | min |
| | time | | |
| | Pressurizing time | 0.5 | min |
| | Loop fill time | 0.2 | min |
| | Loop equilibration | 0.02 | min |
| | time | | |
| | Injection volume | 1 | mL |
| | Injection time | 1 | min |

### Test-Sample Solution

Weigh about 100 mg of sample into 20 mL HS vial, on a balance with 0.01 mg precision and dissolve with 5.0 mL of diluent. Prepare in duplicate.

### TGA Method

TGA analysis was performed under flow of nitrogen (60 ml/min) on TGA 2950 TA instrument, with heating rate of 10° C./min. Standard platinum open pan was used, in temperature range from room temperature to 500° C. Sample mass was about 6-10 mg.

### SEM Method

SEM micrographs are taken on Joel JSM-5800 scanning microscope at 20 kV, WD 20-22, low current. Samples were sputtered with gold by Edwards S150 sputter coater.

### EXAMPLES

### Example 1

### Preparation of Amorphous Fosamprenavir Calcium

Fosamprenavir calcium Form I (0.1 g) was ground in a Fritsch, Pulverisette 7, ball mill. The sample was ground in a 12 mL agate container with 5 agate balls (10 mm diameter) with a speed rate of 700 rpm. Semi-amorphous material was obtained after 15 minutes of grinding. By increasing the time of grinding up to 45 or 60 minutes, the amorphous content increased. After 80 min of grinding, full conversion to the amorphous form occurred. A powder XRD diffractogram of amorphous Fosamprenavir calcium measured after 80 minutes of grinding is shown in FIG. 1.

### Example 2

### Preparation of Amorphous Fosamprenavir Calcium

Fosamprenavir calcium salt, Form I (100 mg) was dissolved in about 10 ml of 3-pentanone in a flask by heating until the suspension became transparent. The resulting solution was cooled down to about 20° C. and poured into a Petri dish. A glassy material was obtained by fast evaporation of 3-pentanone. The material was characterized by DSC as shown in FIG. 3.

### Example 3

### Preparation of Amorphous Fosamprenavir Calcium

Fosamprenavir calcium salt (1.0 g), Form I, was dissolved in methanol (10 mL) and the solution was spray dried under a nitrogen atmosphere. Process parameters: N₂ (40 mm); inlet T=90° C.; outlet T=55-60° C.; pump rate=30% (0.5 L/h); aspirator rate=100%. The white powder obtained by spray drying was analyzed by powder XRD.

### Example 4

### Preparation of Form II of Fosamprenavir Calcium

Fosamprenavir Calcium, Form I (50 mg) was dissolved in N-methyl-2-pyrrolidinone (0.6 ml) at 60° C. The resulting solution was cooled to room temperature and a mixture of water/i-PrOH (0.7 ml/1 ml) was added in one portion. The resulting suspension of crystals was stirred for 45 min, and the product was filtered off, washed with i-PrOH (1 ml) and dried at room temperature, yielding 38 mg of pure Form II.

### Example 5

### Preparation of Form III of Fosamprenavir Calcium

Fosamprenavir Calcium, Form I (50 mg) was placed in a desiccator in an atmosphere of absolute ethanol at 50° C. After 12 days the sample was characterized by powder XRD and a new crystalline form of Fosamprenavir calcium was found.

### Example 6

### Preparation of FSM+Hydroxypropyl Cellulose (HPC)

Fosamprenavir Ca (2.7 g), Form I, was dissolved in methanol (30 mL). HPC (0.27 g) was dissolved in methanol (25 mL). The solutions were combined and the combined solution was spray dried under a nitrogen atmosphere. Process parameters: N2 (40 mm); inlet T=90° C.; outlet T=45-55° C.; pump rate=30% (0.5 L/h); aspirator rate=100%.

### Example 7

### Preparation of FSM+Hydroxypropyl Methyl Cellulose (HPMC)

Fosamprenavir Ca (2.7 g), Form I, was dissolved in methanol (30 mL). HPMC (0.27 g) was dissolved in methanol (35 mL) with heating. The solutions were combined. The combined solution was spray dried under nitrogen atmosphere. Process parameters: N2 (40 mm); inlet T=90° C.; outlet T=45-55° C.; pump rate=30% (0.5 L/h); aspirator rate=100%.

### Example 8

### Preparation of Amorphous Fosamprenavir Calcium

Amorphous Fosamprenavir Ca (15-20 mg) was dissolved in acetone (3 mL) and the filtered solution left at RT. The solid was analyzed by powder XRD.

### Example 9

### Preparation of Amorphous Fosamprenavir Calcium

Amorphous Fosamprenavir Ca (15-20 mg) was suspended in amyl alcohol-mixed isomers (5 mL) and heated to reflux. The hot suspension was filtered and the filtrate left at RT. A solid precipitated and was collected by filtration and analyzed by powder XRD.

### Example 10

### Preparation of Amorphous Fosamprenavir Calcium

Amorphous Fosamprenavir Ca (15-20 mg) was suspended in 2-butanol (5 mL), heated to reflux and filtered. The filtrate was left at RT. A solid precipitated, which was collected by filtration and analyzed by powder XRD.

### Example 11

### Preparation of Amorphous Fosamprenavir Calcium

Amorphous Fosamprenavir Ca (15-20 mg) was suspended in i-butanol (5 mL), heated to reflux and filtered. The filtrate was left at RT. A solid precipitated, which was collected by filtration and analyzed by powder XRD.

### Example 12

### Preparation of Amorphous Fosamprenavir Calcium

Amorphous Fosamprenavir Ca (15-20 mg) was dissolved in 6-fluoro-2-propanol (2.5 mL) and the solution left at RT. A solid precipitate formed and was collected by filtration. The filtered solid was analyzed by powder XRD.

### Example 13

### Preparation of Amorphous Fosamprenavir Calcium

Amorphous Fosamprenavir Ca (15-20 mg) was dissolved in pyridine (2.5 mL) and the solution left at RT. A solid precipitate formed and was collected by filtration. The filtered solid was analyzed by powder XRD.

### Example 14

### Preparation of Rod Like Amorphous Fosamprenavir Calcium

Amorphous Fosamprenavir can be obtained by drying/heating even under normal atmospheric pressure. About 1 g of Fosamprenavir calcium salt, Form I, was placed in Petri dish and put in a heater/oven. After 30 minutes of isothermal heating, the sample was checked and a large proportion of amorphous material was found. Heating was prolonged in order to obtain a sample without crystalline traces. The sample was heated for 1 additional hour at 100° C., 30 minutes at 120° C. and 30 minutes at 130° C. The resulting amorphous material was characterized by powder XRD.

### Example 15

### Preparation of Amorphous Fosamprenavir Calcium

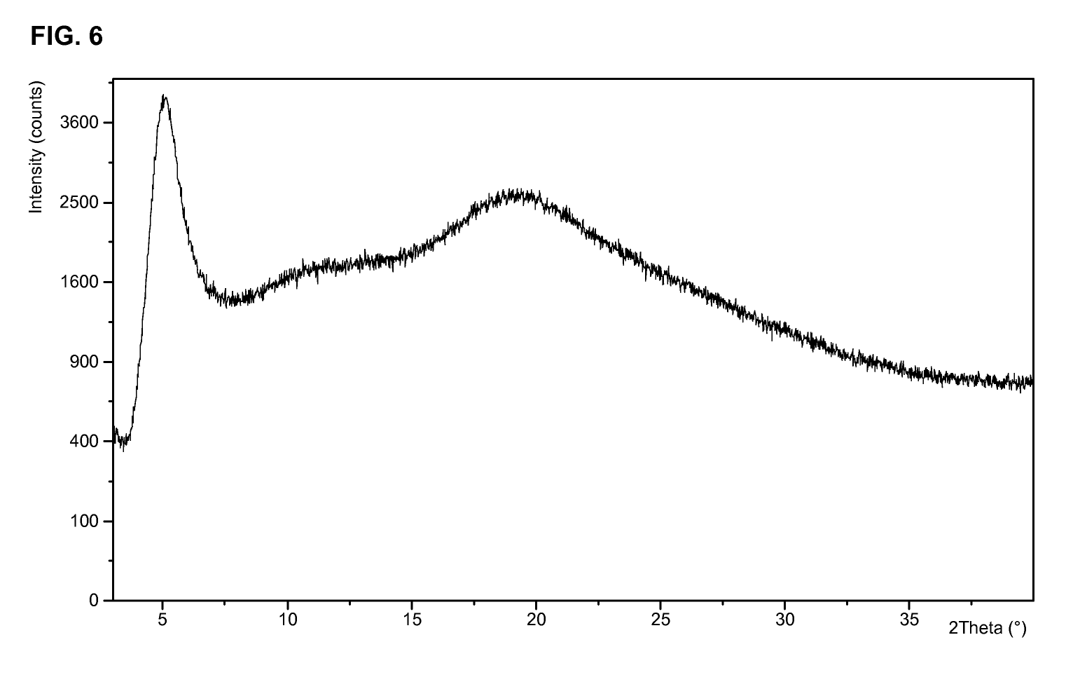

Fosamprenavir calcium, Form P, (30 g) was dissolved in methanol (180 ml) at 25-28° C. 2-Butanol (360 ml) was added dropwise to the stirred solution at 25-28° C. during 75 minutes. A suspension was obtained during the addition (after cca. 100 ml of 2-butanol was added). The suspension was stirred at 25° C. overnight (under a nitrogen stream). A precipitate formed and was filtered off and dried under vacuum at 60° C. for 3 hours. An amorphous powder (24 g) was obtained as characterized by powder XRD (FIG. 6).

### Example 16

### Preparation of Form Iv Acetone Solvate of Fosamprenavir Calcium

Water (4 ml) was added into an acetone solution (29 ml) of fosamprenavir calcium obtained from a hydrogenation process (concentration of fosamprenavir calcium about 85 g/l) at room temperature. After a short time, crystallization started. The resulting suspension was stirred at room temperature for 1 hour. Crystals were filtered off and dried in an open plate at room temperature. 1.4 g of Fosamprenavir calcium, acetone solvate was obtained and characterized by powder XRD. The content of acetone in Fosamprenavir calcium form IV was 4.2% as determined by GC method. Water content measured by KF was 10.9%. TGA solvate/water content was determined by TGA. In the TGA analysis, with heating up to 150° C., the mass lost was 14.4%.

### Example 17

### Preparation of Form IV of Acetone Solvate of Fosamprenavir Calcium

Amorphous Fosamprenavir calcium (2 g) was dissolved in acetone (20 ml) at room temperature. Water (2.7 ml) was added. Very soon crystallization started. The suspension was stirred at room temperature for 15 minutes. Crystals were filtered off and dried in an open plate at room temperature overnight. Fosamprenavir calcium acetone solvate (1.5 g) was obtained and characterized by powder XRD. The water content measured by KF was 11.1%.

### Example 18

### Preparation of Form P of Fosamprenavir Calcium

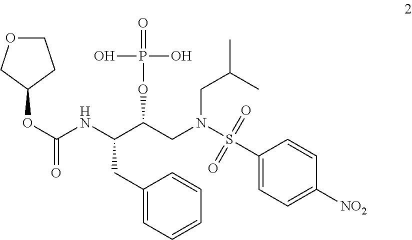

Nitro-Fosamprenavir (Compound 2, 10 g; 16.2 mmol) was dissolved in EtOH (abs.; 100 ml). A hydrogenation catalyst (Merck Pd/C 10%; 0.5 g) was added and the resulting reaction mixture was hydrogenated at hydrogen pressure of 7 bar for 21 hours. The catalyst was then filtered off and the filtrate was concentrated at reduced pressure to approx. 60 ml.

After the solution was heated at 55° C. within 40 min, a solution of CaAc₂×0.5 H₂O (5.4 g; 32.4 mmol; calculated on dry acetate) in water (60 ml) was added dropwise. The resulting solution was cooled to room temperature within one hour, forming a suspension. The suspension was stirred for 2 additional hours, after which the crystalline Ca-salt was filtered off and washed with 50% EtOH (100 ml). The crystalline product was then dissolved in refluxing EtOH (96%, 100 ml). Water (20 ml) was added, after which the mixture was slowly cooled to room temperature. The product which precipitated was then filtered off. The product was dried at room temperature in a vacuum desiccator, yielding 7.97 g of a white powder, crystalline form P, as determined by powder XRD. The content of ethanol was determined by GC method to be 3.2%. The TGA solvate/water content was determined by TGA. With heating up to 150° C., the product lost 13.6%.

### Example 19

### Preparation of Amorphous Fosamprenavir Calcium

Fosamprenavir calcium (form P, 10 g) was suspended in 1-propanol (70 ml). The suspension was heated to about 80° C., at which point a solution was obtained. The solution was cooled down to room temperature. At about 70° C. precipitation occurred. The suspension was stirred at room temperature overnight (for about 15 hours) and the precipitate was then filtered off and dried in vacuum at 80° C. for 24 hours. About 7.2 g of amorphous fosamprenavir calcium was obtained and characterized by powder XRD.

### Example 20

### Preparation of Rod Like Amorphous Fosamprenavir Calcium from Methanol/Water

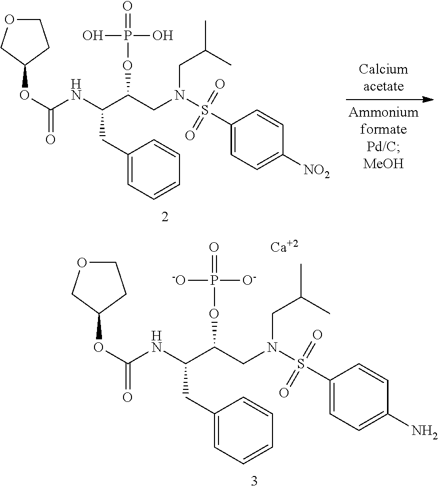

Nitro-Fosamprenavir (2) (15.0 g; 24.5 mmol) and calcium acetate (4.24 g, 26.7 mmol) were suspended in methanol (180 ml) in a 500 ml round bottom flask. After 5 minutes ammonium formate (6.2 g; 97.5 mmol) and 10% Pd/C (960 mg, 3% w/w) were added and the reaction mixture was heated at 65° C. for 90 minutes. After the reaction was finished, the warm reaction mixture was filtered through Celite to remove the Pd/C catalyst and the filter cake was rinsed with methanol (2×30 ml). To the warm filtrate (50° C., 250 ml) water (62.5 ml) was added dropwise forming a suspension which was further stirred at room temperature overnight. The product (3, form I), a white solid, was filtered off and heated under vacuum at 45° C. for 3 hours and additionally 5 hours at 100° C. and 2 hours at 120° C.

### Example 21

### Preparation of Rod Like Amorphous Fosamprenavir Calcium from Methanol-Ethanol/Water

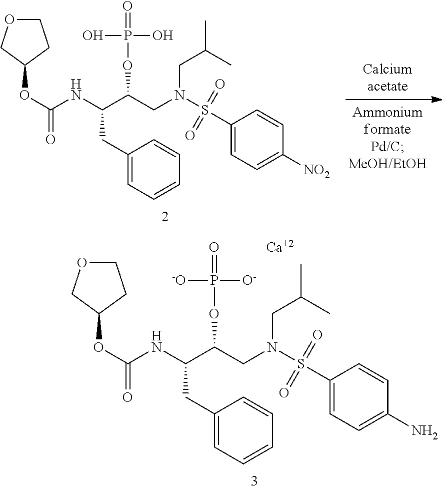

Nitro-Fosamprenavir (2) (15.0 g; 24.5 mmol) and calcium acetate (4.24 g, 26.7 mmol) were suspended in a solvent mixture (methanol 70 ml and ethanol 110 ml) in a 500 ml round bottom flask. After 5 minutes, ammonium formate (6.2 g; 97.5 mmol) and 10% Pd/C (960 mg, 3% w/w) were added and the reaction mixture was heated at 65° C. for 90 minutes. After the reaction was finished, the warm reaction mixture was filtered through Celite to remove the Pd/C catalyst and the filter cake was rinsed with methanol (30 ml). To the warm filtrate (50° C., 210 ml) water (40 ml) was added dropwise. The resulting mixture was cooled to room temperature and a white suspension was obtained. The suspension was stirred at room temperature overnight. The product (3, form I), a white solid, was filtered off and heated under vacuum at 45° C. for 3 hours and additionally 5 hours at 100° C. and 2 hours at 120° C.

### Example 22

### Preparation of Rod Like Amorphous Fosamprenavir Calcium from 1-Propanol

Fosamprenavir calcium (form I) is suspended in 1-propanol (70 ml) at room temperature. The suspension is then heated to 75-80° C. to dissolve. The solution is then cooled down to room temperature. Precipitation starts at about 70° C. forming a suspension. The suspension is stirred at room temperature for additional 2 hours. The precipitate is filtered off and dried at reduced pressure (70-300 mbar) under nitrogen stream overnight and then additional 5 hours at 100° C. in vacuum. Amorphous fosamprenavir calcium is obtained.

What is claimed is:

1. A rod like amorphous form of Fosamprenavir calcium salt.

2. The rod like amorphous form of Fosamprenavir calcium salt of claim 1, characterized by data selected from: a SEM image as depicted in FIG. 10, a SEM image as depicted in FIG. 11; a SEM image as depicted in FIG. 12, a SEM image as depicted in FIG. 13, and combinations thereof.

3. The rod like amorphous form of Fosamprenavir calcium salt of claim 1, obtainable by a process comprising heating Fosamprenavir calcium salt, form I, having XRPD peaks at 5.735, 9.945, 11.500, 13.780, 14.930, 15.225, 17.980, 19.745, 21.575, 22.170, 24.505 and 27.020° 2θ±0.2° 2θ.

4. The rod like amorphous form of Fosamprenavir calcium salt of claim 1, obtainable by the process of Example 20, Example 21 or Example 22.

5. A pharmaceutical composition comprising a rod like amorphous form of Fosamprenavir calcium salt according to claim 1, and at least one pharmaceutically acceptable excipient.

6. A rod like amorphous form according to claim 1, for use in treating HIV.

7. A method of preparing a rod like amorphous form of Fosamprenavir calcium salt, comprising heating Fosamprenavir calcium salt, form I, having XRPD peaks at 5.735, 9.945, 11.500, 13.780, 14.930, 15.225, 17.980, 19.745, 21.575, 22.170, 24.505 and 27.020° 2θ±0.2° 2θ until a rod like amorphous form of Fosamprenavir calcium salt is formed.

8. A method of treating a patient infected with HIV, comprising administering the pharmaceutical composition of claim 5.
